(19) United States
(12) Patent Application Publication
(10) Pub. No.: US 2020/0246198 A1
(43) Pub. Date: Aug. 6, 2020

(54) CLEANING AND DISINFECTING STICK FOR PIERCED HOLES IN THE SKIN IN WHICH JEWELRY AND PIERCINGS ARE INSERTED AND WORN

(71) Applicant: Jlena PECORARO, Gross (CH)

(72) Inventor: Jlena PECORARO, Gross (CH)

(21) Appl. No.: 15/769,866

(22) PCT Filed: Oct. 18, 2016
(86) PCT No.: PCT/EP2016/074975

§ 371 Date: Apr. 20, 2018

(30) Foreign Application Priority Data
Oct. 20, 2015 (CH) 01525/15

(51) Int. Cl. A61F 13/38; A46B 3/00; A46B 9/00; A46B 15/00; A61F 11/00

(52) U.S. Cl. CPC A61F 13/38; A46B 3/005; A46B 2200/10; A46B 15/0069; A61F 11/006; A46B 9/005

(57) ABSTRACT

Cleaning and disinfection stick is designed in the manner of a nylon toothpick. In contrast to a toothpick, it is arcuate over its length, wherein straight embodiments are also possible. It tapers from its remaining diameter into a fine tip in at least one of its ends regions. Adjacent to the fine tip, the stick can include a round brush. Alternatively, it can also include a felt-like, textile or fluffy surface or be provided with a rod-like paper or plastic attachment. After removing a piercing, it can be inserted, leading with the fine tip into the skin hole and subsequently be pushed further into the skin hole until its cleaning body is inserted therein. It is then gently moved to and fro or rolled to and fro about its axis and pulled out again.

The present invention relates to a cleaning and disinfection stick, in order with this, to clean pierced holes in the skin which are pierced for inserting jewelry or piercings

Piercing holes are pierced at different locations of the body. Most widespread are holes in the earlobes which are used for inserting jewelry, fashion jewelry, creoles and earrings. However, the nasal wings, the upper or lower lip, the skin in the region of the eyebrows or on the bellybutton are also pierced, and piercings are even worn on the private parts.

Everyone who wears a piercing or jewelry in such skin holes knows the smell when these secrete, above all when one pulls the jewelry or the piercing out of the skin holes and these are then free and open. This smell is penetrative, putrid and very unpleasant. Until now there have been no targeted methods, in order to systematically clean such piercings or skin holes, in order to prevent infections and to prevent the mentioned unpleasant smell and to treat or prevent infections. Jewelry and piercings on the ears are regularly removed before going to sleep, so that they no longer disturb when one lies with one's head on the side. Noses and other facial piecing are likewise removed, if possibly not on a daily basis, then regularly, in order to clean the skin as best as possible. Until now however, it was only the piercing/metal itself which could be properly cleaned and disinfected, but the skin holes could not be systematically and thoroughly cleaned until now.

For this, a disinfectant is conventionally merely wiped over the skin hole and as a makeshift is rubbed in somewhat with a paper handkerchief. However, there is no targeted cleaning possibility for the skin holes and their thorough disinfection is also lacking.

It is the object of this invention to provide a cleaning and disinfection stick for this, so that the skin holes can be thoroughly and comfortably cleaned and can hereby be thoroughly disinfected, not least in order herewith to avoid the very unpleasant odours which otherwise occur.

This object is achieved by a cleaning and disinfection stick in the manner of a nylon toothpick which is characterised in that firstly, at least in one of its end regions, it tapers from its remaining diameter into a bare tip, and secondly, shortly after this bare tip, forms a round brush or there has a felt-like, textile or fluffy surface or is provided with a rod-like or tube-like paper or plastic attachment.

Various embodiments of this cleaning and disinfection stick are presented in more detail and their handling and action described in the subsequent description by way of the drawings.

There are shown in:

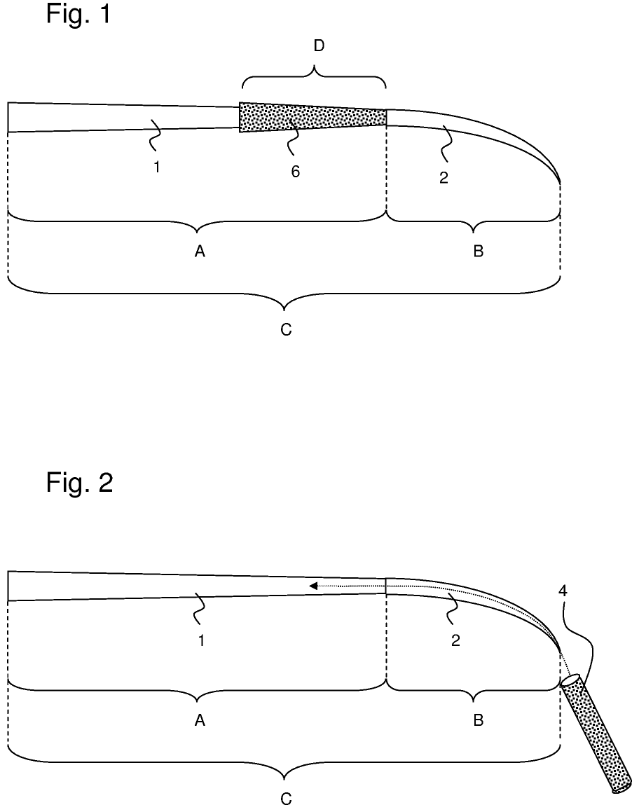

FIG. 1: a cleaning and disinfection stick which at the front tapers into an arcuate, bare tip with a cleaning part on the straight part of the disinfection stick;

FIG. 2: a cleaning and disinfection stick which at the front runs out into an arcuate, bare tip, with a cleaning or disinfection tube which can be stuck on from the tip;

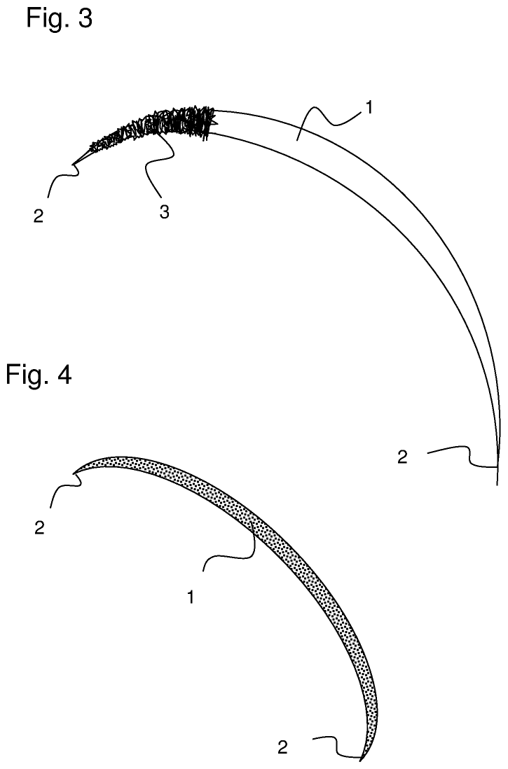

FIG. 3: a cleaning and disinfection stick with a sickle-shaped curvature and with a round brush in its one end region, specifically in its front part, wherein this end region tapers into a free tip:

FIG. 4: a cleaning and disinfection stick with a sickle-like curvature and with a felt-like or textile-like coating;

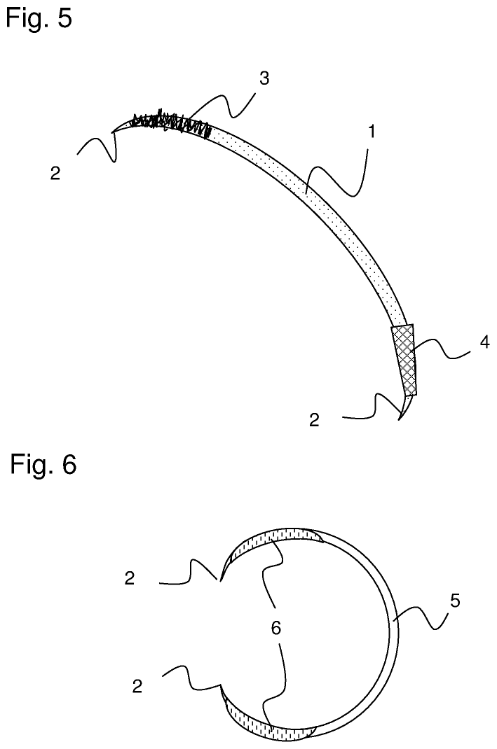

FIG. 5: a cleaning and disinfection stick with a sickle-like curvature and with a round brush at its one end region and with a felt-like or textile-like coating at its other end region;

FIG. 6: a circular cleaning and defection stick with an interrupted circle whose two end regions each comprise a textile-like or felt-like coating and taper into a tip.

FIG. 1 shows a preferred embodiment of the cleaning and disinfection stick of plastic. Here, it consists of a straight grip stick 1 which extends over the region A. At the front, an arcuate, bare tip 2 connects onto this grip stick 1 over a region B. This tip 2 begins from the grip stick 1 with a diameter of approx 2 mm and runs over a length of approx. 2 cm into a tip which is designed as sharply as possible, but at the very front has a thickness of 0.5 mm at the most. The grip stick 1 is a rod initially with a diameter of approx 5 mm and which is tapered to the front over the length of the grip stick 1, which is to say over its length of approx 4 cm, to approx 2 mm diameter. The cleaning and disinfection stick obtains an adequate stability on account of this, and the front tip 2 simultaneously permits the secure and gentle introduction of the cleaning and disinfection stick into a piercing hole, since this tip 2 at its frontmost region yields in a soft-elastic manner and becomes stiffer to the rear on account of the continuous thickening. In the front region of the grip stick 1, this forms a felt-like, textile or fluffy surface 6, as is shown in FIG. 1.

For application, the cleaning and disinfection stick is gripped on the grip stick 1 between the thumb and the index finger and with its bare tip 2 is inserted into the skin hole of a previously removed piercing. Thereby, it is also clear as to why the tip 2 must be designed in such a sharp and moreover relatively soft manner. The piercing holes are usually very fine. Indeed they often grow to close in the case that a piercing has not been used for a long time. One succeeds in introducing the cleaning and disinfection stick into the piercing hole due to the fine tip 2. If one does not hit the hole, then the tip 2 is bent due to its softness, so that the danger of injury is avoided. As soon as the tip 2 of the cleaning and disinfection stick has been inserted into a piercing hole, it acts as a guide and the cleaning and disinfection stick can herewith be inserted further into the hole, so that its surface 6 which here is felt-like, textile or fluffy is pushed into the hole here. It can be pushed into the hole to a greater or lesser extent depending on the width of the skin hole. If necessary, it can be rotated in the hole somewhat, by way of the grip stick 1 being rolled to and fro between the thumb and index finger of the user or being moved to and fro in the axial direction. The efficient cleaning of the piercing hole is achieved with this. A round brush can take the place of a felt-like, textile or fluffy surface. This region D for cleaning can also be impregnated with a disinfectant, so that apart from the cleaning, an efficient disinfection of the piercing hole is also achieved.

In an alternative as is shown in FIG. 2, the grip stick 1 is provided with a tube-like paper or plastic attachment 4 which from the tip 2 and from the front can be stuck over the rearwardly conically widening grip stick 1 as is drawn by a long arrow and can therefore be fixedly stuck onto it. The tip 2 can be correspondingly elastically bent for this, so that the tube brushes over it and can be pushed to the rear until it firmly clamps on the grip stick 1.

As a further variant, this cleaning and disinfecting stick can be designed as a round brush in the front region of the grip stick 1, by way of the plastic there being accordingly injected with radial bristles or a separately manufactured tube with radial bristles or a fluffy outer side being stuck onto the grip stick 1 from the front via the tip 2.

FIG. 3 shows a further embodiment of the cleaning and disinfection stick. In principle, it is designed very similarly to a nylon toothpick, with a brush at the front end. In contrast to these toothpicks, the cleaning and disinfection stick is arcuate over its complete length and here forms a thin sickle shape. It can be designed more thickly in the middle region as a grip stick 1 and be shaped in a manner tapering towards the ends, but it can also be designed equally thickly over almost the entire curvature. At all events, it tapers into a very fine, bare point or tip at least at its one end. In the shown example, both ends taper into bare tips 2. These bare tips 2 are very sharp, thus for example taper into the tip at a very acute angle over approx. 1 to 2 mm. However, the tip is relatively soft, since the cleaning and disinfection stick is manufactured of a suitable plastic, preferably of the same or similar plastic as nylon toothpicks. In the front region of this sharp, bare end, the cleaning and disinfection stick is provided with a round brush 3 which is likewise designed in a very thin manner, even thinner than with a nylon toothpick, and its round brush is approx 1 mm thick at the front and approx. 2 mm thick at the end. A round brush of between 0.5 to 1.5 mm diameter is suitable, wherein these dimensions can also differ. In front of the round brush 3, the tip 2 must conically taper from a thickness of approx 0.5 mm to 1 mm over a 2 mm to 5 mm length into a point and be free and bare. Of course, such cleaning sticks which carry a round brush at both ends are also possible, or at one end a round brush and at the other end a special textile-like, felt-like or fluff-like or fluffy coating, in order to achieve a different cleaning effect by way of this

FIG. 4 shows a cleaning and disinfection stick 1 of a similar shape, but which is provided with a textile-like, felt-like or fluffy like or fleecy coating over its entire body. It can be used just as the previously described variants.

FIG. 5 shows a sickle-shaped cleaning and disinfection stick whose one end carries a cleaning brush, whilst a conically tapering, felt-like attachment 4 is attached on its other end, for instance welded on, bonded on or simply stuck on in a mechanically clamping manner. This cleaning and disinfection stick 1 can be used arbitrarily firstly with the brush then with the felt rod or in a reverse sequence.

FIG. 6 shows a part-ring-shaped cleaning and disinfection stick 5. A part of the ring, roughly a quarter of its circumference or more is absent, so that two ends 2 are formed and these are each provided with a round brush, or as is shown here, with a coating which is particularly suitable for the cleaning, for example something felt-like, textile-like or fluffy. As an alternative, paper tubes can also be pushed over the sharp end regions until the tip appears at the other side, or continuous paper plugs (small plugs or cones) or rods (small rods) can be stuck onto the sharp end regions in the axial direction, until the tip of the cleaning and disinfection stick appears out of the paper plug or rod at the other side.

The ends with the cleaning bodies—irrespective of the material from which these cleaning bodies consist—can be impregnated or sprayed with disinfectant. Variants with different suitable disinfectants can also be offered.

Basically, at least the tips of the cleaning sticks must be arcuate, so that the piercing holes can be accessed. It is particularly with eyebrow piercings or bellybutton piercings that the access and the pushing-through with a straight cleaning stick is quite difficult or not possible at all. For this reason, it is important for at least one end to run out into an arcuate, very fine but not too hard tip, so that the cleaning and disinfection stick can also be stuck into the smallest of skin holes. As soon as the tip has once been introduced into a skin hole, the cleaning and disinfection stick can then be pushed after this, until it is inserted with its cleaning body, thus for instance the round brush, the cleaning coating or the cleaning plug or tube, into the skin or the piecing hole. It is then gently moved forwards and backwards therein or is rolled to and fro about its axis, whilst leaving behind some disinfectant.

The cleaning and disinfection stick can be arcuate at constant radii or with increasing radii, or also form a part-ring or a part-ellipse. Its regions which serve as effective cleaning surfaces can be a round brush which is injected onto the cleaning and disinfection stick for example as a second injection moulding component of plastic. The end regions can also be coated with a special material or be deposited by an immersion bath, so that they feel like textile material, such as felt or are fluffy. Finally, cleaning rods, such as paper tube or paper plugs can be stuck, welded or bonded onto the end regions. These can run in a straight manner with a constant diameter, or they can also be designed as conically tapering tubes or rods.

Unpleasant odours can be eliminated after the removal of a piecing with such a cleaning of the piercing holes, irrespective of the body location. Infections can be avoided thanks to the simultaneously executed disinfection of the skin hole, or if such occurs, then these can be effectively treated.

### LIST OF REFERENCE NUMERALS

1 grip stick
2 tip
3 round brush
4 rod-like or tube-like paper or plastic attachment
5 part-ring-shaped cleaning and disinfection stick
6 textile-like, felt-like or fluffy material

1. A cleaning and disinfection stick of a material as with a nylon toothpick, the cleaning and disinfection stick comprising:
a grip stick and that this firstly at least in one of its end regions is designed in an arcuate manner, secondly tapers from its thickest diameter into a bare, fine tip which on account of its fineness is soft-elastic and thirdly, in the reverse direction, seen from its tip, shortly after this bare tip forms a round brush or comprises a felt-like, textile or fluffy surface there or is provided there with a rod-like or tube-like attachment.

2. The cleaning and disinfection stick according to claim 1, wherein the cleaning and disinfection stick has a curvature over its entire length or at least from the rear end of the tip to its front end.

3. The cleaning and disinfection stick according to claim 1, wherein the round brush consists of plastic and is injected onto the plastic stick as a second injected component.

4. The cleaning and disinfection stick according to claim 1, wherein the complete stick is coated with a textile-like, felt-like or fluffy material, for forming a textile-like, felt-like or fluffy surface.

5. The cleaning and disinfection stick according to claim 1, wherein at least at one end it comprises a rod-like or tube-like attachment of paper, natural, pressed fibre material or plastic, which as a tube or plug is stuck onto the end region to such an extent that the bare, fine tip of the cleaning stick appears out of the tube or plug or rod at the other side.

6. The cleaning and disinfection stick according to claim 1, wherein the cleaning and disinfection stick forms a part-ring, whose two end regions each taper into a bare, fine tip which are envisaged for insertion into a piercing hole.

7. The cleaning and disinfection stick according to claim 1, wherein the region or regions which are envisaged to act as cleaning bodies are impregnated or sprayed with a disinfectant and the cleaning and disinfection stick is packaged into a compact individual package.

8. The cleaning and disinfection stick according to claim 1, wherein the region or regions which are envisaged to act as cleaning bodies are impregnated or sprayed with a disinfectant.

9. A cleaning and disinfection stick comprising:
a grip portion;
a first end portion and a second end portion extending from opposing sides of the grip portion, at least one of the first or second end portions being defined as a portion that tapers from a diameter corresponding to a substantial majority of the grip portion to a fine tip and having an arcuate shape; and
an attachment coupled to at least one of the first or second end portions, the attachment being one of a round brush, a felt-like textile, a fluffy surface, and/or a rod-shaped attachment.

10. The cleaning and disinfection stick according to claim 9, wherein the grip portion has a curvature over its entire length.

11. The cleaning and disinfection stick according to claim 9, wherein the grip portion has a curvature from an end of the first or second end portion to the opposing one of the first or second end portions.

12. The cleaning and disinfection stick according to claim 9, wherein the round brush includes a plastic and is injected onto the grip portion as an injected component.

13. The cleaning and disinfection stick according to claim 9, wherein the grip portion is coated with a textile-like, felt-like, or fluffy material.

14. The cleaning and disinfection stick according to claim 9, wherein the attachment is the rod-shaped attachment, the rod-shaped attachment being made of at least one of paper, a fibrous material, or plastic, which is coupled with the at least one of the first or second end portions such that the fine tip of the gripping portion extends out of the attachment.

15. The cleaning and disinfection stick according to claim 9, wherein the grip portion and the first and second end portions form a partial ring shape, wherein the fine tip is operable to be inserted into a piercing hole.

16. The cleaning and disinfection stick according to claim 9, wherein at least one of the first or second portions is covered with a disinfectant.

17. The cleaning and disinfection stick according to claim 9, wherein the cleaning and disinfection stick is packaged into an individual package.

18. The cleaning and disinfection stick according to claim 9, wherein the fine tip is flexible.
